(19) United States
(12) Patent Application Publication
(10) Pub. No.: US 2026/0088174 A1
(43) Pub. Date: Mar. 26, 2026

(54) METHOD OF ESTABLISHING MODEL AND METHOD FOR DETECTING ATRIAL FIBRILLATION AND SLEEP APNEA

(71) Applicant: National Cheng Kung University, Tainan City (TW)

(72) Inventors: Che-Wei LIN, Tainan City (TW); FEBRYAN SETIAWAN, Tainan City (TW); Cheng-Yu LIN, Tainan City (TW)

(21) Appl. No.: 19/337,375

(22) Filed: Sep. 23, 2025

Related U.S. Application Data
(60) Provisional application No. 63/698,728, filed on Sep. 25, 2024.

(51) Int. Cl. G16H 50/20; A61B 5/00; A61B 5/361; G06F 18/213; G06N 3/09; G16H 10/60; G16H 50/30

(52) U.S. Cl. CPC G16H 50/20; A61B 5/361; A61B 5/4818; G06F 18/213; G06N 3/09; G16H 10/60; G16H 50/30

(57) ABSTRACT

A method of establishing a model for detecting atrial fibrillation (AF) and sleep apnea (SA) is implemented by a computer system that stores training electrocardiograms, and includes steps of: dividing the training electrocardiograms into training segments, each of which contains a common length of time of recorded electrical activity of a heart; for each of the training segments, labeling the training segment with a symptom indicator that indicates whether the training segment is related to AF and whether the training segment is related to SA; for each of the training segments, performing feature extraction on the training segment to obtain an entry of feature data; and establishing the model by using a machine learning algorithm based on the entries of feature data and the symptom indicators.

### CROSS-REFERENCE TO RELATED APPLICATION

This application claims the benefit of U.S. Provisional Patent Application No. 63/698,728, filed on Sep. 25, 2024, the entire disclosure of which is incorporated by reference herein.

### FIELD

The disclosure relates to a method for detecting atrial fibrillation (AF) and sleep apnea (SA), and a method of establishing a model for detecting AF and SA.

### BACKGROUND

Obstructive sleep apnea (OSA) is a common sleep-related breathing disorder. While a patient with OSA sleeps, his/her upper-airway is partially or completely obstructed, leading to repeated episodes of reduced or absent breathing during sleep, i.e., apnea hypopnea (AH). OSA is attributable to reduced upper airway muscle tone, and over-relaxed, excess or enlarged soft tissues in the pharynx of the patient with OSA.

Atrial fibrillation (AF) is an abnormal heart rhythm (i.e., arrhythmia) characterized by an irregular and rapid beating of the atrial chambers of the heart, and may cause an increased risk of heart failure. It is worthy of note that OSA is one of multiple risk factors of AF. In other words, OSA increases the risk and progression of AF. In particular, patients with OSA are four times more likely than those without OSA to experience AF.

Because of population ageing and increased incidence rates of obesity, hypertension, metabolic syndromes and cardiovascular diseases, OSA and AF are becoming more widespread. OSA and AF have common risk factors and medical complications, but treatments for OSA and AF are different. Specifically, treatments for OSA exemplarily include losing weight, using CPAP (Continuous Positive Airway Pressure), surgical therapy and so on; treatments for AF exemplarily include medication, electrical cardioversion, radiofrequency ablation (RFA) and so on.

### SUMMARY

Therefore, an object of the disclosure is to provide a method of establishing a model for detecting atrial fibrillation (AF) and sleep apnea (SA), and a method for detecting AF and SA.

According to one aspect of the disclosure, the method of establishing a model is adapted to be implemented by a computer system that stores multiple training electrocardiograms. The method includes steps of:
dividing each of the training electrocardiograms into multiple training segments, each of which contains a common length of time of recorded electrical activity of a heart;
for each of the training segments derived from each of the training electrocardiograms, labeling, based on a user input, the training segment with a symptom indicator that indicates whether the training segment is related to AF and whether the training segment is related to SA;
for each of the training segments derived from each of the training electrocardiograms, performing feature extraction on the training segment to obtain an entry of feature data; and
establishing the model for detecting AF and SA by using a machine learning algorithm based on the entries of feature data obtained respectively from the training segments that are derived from each of the training electrocardiograms and the symptom indicators labelled respectively on the training segments that are derived from each of the training electrocardiograms.

According to another aspect of the disclosure, the method for detecting AF and SA is adapted to be implemented by a computer system that stores a model for detecting AF and SA. The method includes steps of:
obtaining a target electrocardiogram that is related to a subject;
dividing the target electrocardiogram into multiple target segments, each of which contains a common length of time of recorded electrical activity of a heart of the subject;
for each of the target segments derived from the target electrocardiogram, performing feature extraction on the target segment to obtain an entry of feature data; and
for each of the target segments, obtaining a symptom prediction result by using the model for detecting AF and SA based on the entry of feature data obtained from the target segment, the symptom prediction result indicating whether the target segment is related to AF and indicating whether the target segment is related to SA.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a block diagram illustrating a computer system according to an embodiment of the disclosure.

FIG. 2 is a flow chart illustrating a method of establishing a model for detecting atrial fibrillation (AF) and sleep apnea (SA) according to an embodiment of the disclosure.

FIG. 3 is a flow chart illustrating a method for detecting AF and SA according to an embodiment of the disclosure.

### DETAILED DESCRIPTION

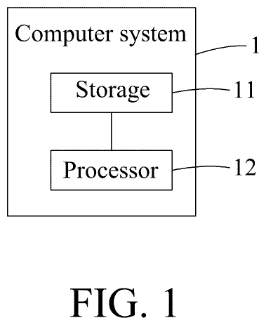

Referring to FIG. 1, an embodiment of a computer system 1 according to the disclosure is illustrated. The computer system 1 may be implemented to be a desktop computer, a laptop computer, a notebook computer or a tablet computer, but implementation thereof is not limited to what are disclosed herein and may vary in other embodiments.

The computer system 1 includes a storage 11, and a processor 12 that is electrically connected to the storage 11.

The processor 12 may be implemented by a central processing unit (CPU), a microprocessor, a micro control unit (MCU), a system on a chip (SoC), or any circuit configurable/programmable in a software manner and/or hardware manner to implement functionalities discussed in this disclosure.

The storage 11 may be implemented by random access memory (RAM), double data rate synchronous dynamic random access memory (DDR SDRAM), read only memory (ROM), programmable ROM (PROM), flash memory, a hard disk drive (HDD), a solid state disk (SSD), electrically-erasable programmable read-only memory (EEPROM) or any other volatile/non-volatile memory devices, but is not limited thereto.

In one embodiment, the storage 11 stores multiple training electrocardiograms. The training electrocardiograms are obtained from the Sleep Heart Health Study visit 1 (SHHS1) database, and include overnight single-lead ECG recordings of 101 participants. Thirty six (36) of the 101 participants were diagnosed with prevalent atrial fibrillation (PAF), 25 of the 101 participants were diagnosed with incident atrial fibrillation (IAF), and 40 of the 101 participants were diagnosed with obstructive sleep apnea (OSA).

In one embodiment, the storage 11 stores a model for detecting atrial fibrillation (AF) and sleep apnea (SA) that has been trained based on the training electrocardiograms. It should be noted that SA to be detected by using the model exemplarily includes OSA, and AF to be detected by using the model exemplarily includes PAF and IAF. The model for detecting AF and SA is a lightweight deep learning neural network model, and has an input layer, sixteen convolution layers, fifteen batch normalization layers, fifteen leaky rectified linear unit (ReLU) activation layers, five max pooling layers, a global average pooling (GAP) layer, a softmax layer and an output layer.

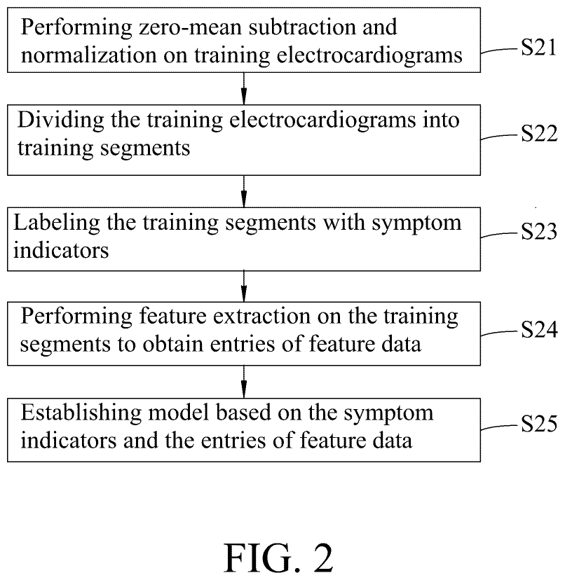

Referring to FIG. 2, an embodiment of a method of establishing a model for detecting AF and SA according to the disclosure is illustrated. The method is adapted to be implemented by the computer system 1 that stores multiple training electrocardiograms. The method includes steps S21 to S25 delineated below.

In step S21, the processor 12 of the computer system 1 performs zero-mean subtraction and normalization on the training electrocardiograms.

In step S22, after zero-mean subtraction and normalization have been performed on the training electrocardiograms in step S21, the processor 12 divides each of the training electrocardiograms into multiple training segments, each of which contains a common length of time of recorded electrical activity of a heart. The common length of time is exemplarily 60 seconds, but is not limited thereto.

In step S23, for each of the training segments derived from each of the training electrocardiograms, the processor 12 labels, based on a user input (e.g., which may be generated according to user operations performed on a keyboard of the computer system 1), the training segment with a symptom indicator that indicates whether the training segment is related to AF and whether the training segment is related to SA. Specifically, for each of the training segments derived from each of the training electrocardiograms, the symptom indicator indicates one of a first condition that the training segment is related to both AF and SA (hereinafter also referred to as the “AF AH” class), a second condition that the training segment is related to AF but not to SA (hereinafter also referred to as the “AF no-AH” class), a third condition that the training segment is related to SA but not to AF (hereinafter also referred to as the “no-AF AH” class), and a fourth condition that the training segment is related to neither AF nor SA (hereinafter also referred to as the “no-AF no-AH” class). It is worthy of note that the training segments labelled with the “AF AH” and “AF no-AH” classes may be derived from the training electrocardiograms corresponding to patients with PAF; the training segments labelled with the “AF AH”, “AF no-AH”, “no-AF AH” and “no-AF no-AH” classes may be derived from the training electrocardiograms corresponding to patients with IAF; the training segments labelled with the “AF AH”, and “no-AF AH” classes may be derived from the training electrocardiograms corresponding to patients with OSA.

In step S24, for each of the training segments derived from each of the training electrocardiograms, the processor 12 performs feature extraction on the training segment to obtain an entry of feature data. Specifically, in this embodiment, for each of the training segments derived from each of the training electrocardiograms, the processor 12 performs reconstruction independent component analysis (RICA) on the training segment. In particular, a number of independent components (ICs) is set to be 500 in RICA.

In step S25, the processor 12 establishes the model for detecting AF and SA by using a machine learning algorithm based on the entries of feature data obtained respectively from the training segments that are derived from each of the training electrocardiograms, and based on the symptom indicators labelled respectively on the training segments that are derived from each of the training electrocardiograms. In particular, the machine learning algorithm used to establish the model for detecting AF and SA is the DarkNet algorithm. By virtue of the architecture of the model, performances of the model for detecting AF and SA that has been trained are relatively improved and are exemplarily shown in Table 1 below. Since metrics (including accuracy, sensitivity, specificity, F1 score, and the area under the curve, AUC) for evaluating a diagnostic test have been well known to one skilled in the relevant art, detailed explanation of the same is omitted herein for the sake of brevity.

TABLE 1

| Class | Accuracy | Sensitivity | Specificity | F1 score | AUC |
|---|---|---|---|---|---|
| AF AH | 99.7 | 99.3 | 99.8 | 99.3 | 0.9999 |
| AF no-AH | 97 | 96.6 | 97.2 | 96.2 | 0.9957 |
| No-AF AH | 99.7 | 98.7 | 99.9 | 98.9 | 0.9999 |
| No-AF no- | 97 | 93.9 | 98.2 | 94.4 | 0.9948 |
| AH | | | | | |
| Average | 98.4 | 97.1 | 98.8 | 97.2 | 0.9973 |

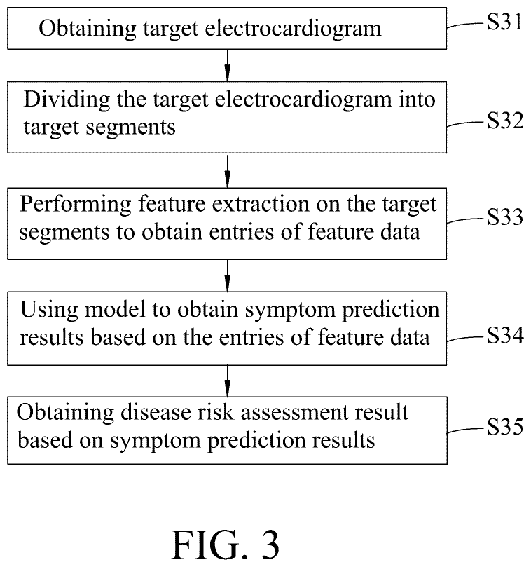

Referring to FIG. 3, an embodiment of a method for detecting AF and SA according to the disclosure is illustrated. The method is adapted to be implemented by the computer system 1 that stores the model for detecting AF and SA. The method includes steps S31 to S35 delineated below.

In step S31, the processor 12 obtains a target electrocardiogram that is related to a subject (e.g., a human). The target electrocardiogram is exemplarily obtained by performing overnight single-lead ECG on the subject.

In step S32, the processor 12 divides the target electrocardiogram into multiple target segments, each of which contains the common length of time (i.e., 60 seconds) of recorded electrical activity of a heart of the subject.

In step S33, for each of the target segments derived from the target electrocardiogram, the processor 12 performs feature extraction on the target segment to obtain an entry of feature data. Specifically, the processor 12 performs RICA on the target segment.

In step S34, for each of the target segments, the processor 12 obtains a symptom prediction result by using the model for detecting AF and SA based on the entry of feature data obtained from the target segment. The symptom prediction result indicates whether the target segment is related to AF and indicates whether the target segment is related to SA. In particular, the processor 12 classifies the target segment into one of a first medical condition which is related to both AF and SA, a second medical condition which is related to AF but not to SA, a third medical condition which is related to SA but not to AF, and a fourth medical condition which is related to neither AF nor SA. Then, the processor 12 obtains the symptom prediction result that indicates one of the first to fourth medical conditions into which the target segment is classified.

In step S35, the processor 12 obtains a disease risk assessment result for the subject based on the symptom prediction results respectively of the target segments. The disease risk assessment result indicates whether the subject is at risk of having AF and indicates whether the subject is at risk of having SA. Specifically, for each of the first to fourth medical conditions, the processor 12 counts a number of the target segments that are classified into the medical condition so as to obtain four numbers respectively for the first to fourth medical conditions. Subsequently, the processor 12 selects one of the first to fourth medical conditions that corresponds to a greatest one of the four numbers, and obtains the disease risk assessment result to indicate said one of the first to fourth medical conditions thus selected.

In some embodiments, after step S35, the processor 12 outputs the disease risk assessment result by printing the disease risk assessment result on paper(s), displaying the disease risk assessment result on a screen, and so on.

In some embodiments, after outputting the disease risk assessment result, the processor 12 further generates and outputs treatment recommendations based on the disease risk assessment result. For example, when the disease risk assessment result indicates that the subject is at risk of having AF but not SA, the processor 12 would generate and output the treatment recommendation that the subject could be treated with electrical cardioversion or radiofrequency ablation (RFA). When the disease risk assessment result indicates that the subject is at risk of having SA but not AF, the processor 12 would generate and output the treatment recommendation that the subject could be treated with CPAP (Continuous Positive Airway Pressure) or otolaryngological surgery (also known as ENT surgery or otolaryngology-head and neck surgery). It is worthy of note that typically, a treatment for AF is rarely carried out together with a treatment for OSA. At the same time, some studies have indicated that providing a treatment for OSA to OSA patients who are also diagnosed with AF could simultaneously cure both SA and AF with a relatively high probability. Therefore, when the disease risk assessment result indicates that the subject is at risk of having both SA and AF, the processor 12 would generate and output the treatment recommendation that the subject could be treated with CPAP or otolaryngological surgery for addressing SA first.

It should be noted that in some embodiments, the computer systems 1 for implementing the method of establishing a model and the method for detecting AF and SA are the same, i.e., a single one computer system 1 is used to implement both the method of establishing a model and the method for detecting AF and SA. In some embodiments, the computer systems 1 for implementing the method of establishing a model and the method for detecting AF and SA are separate, i.e., one of the computer systems 1 is used to implement the method of establishing a model, and another of the computer systems 1 is used to implement the method for detecting AF and SA.

To sum up, for the method for detecting AF and SA according to the disclosure, a neural network model for detecting AF and SA is established first. Thereafter, by utilizing the model thus established, it is possible to determine, based only on an electrocardiogram obtained from overnight single-lead ECG performed on a subject, whether AF or SA ever occurred when the subject slept, and then to further determine whether a subject is at risk of having AF and whether the subject is at risk of having SA. In this way, medical professionals may be able to provide the subject with suggestions and treatments for AF or SA. In addition, using a neural network model to detect AF and SA based on an electrocardiogram may save hardware costs on expensive instruments and equipment, may save space for installation of instruments and equipment, and may lower a barrier to entry for operators of instruments and equipment. Moreover, it would be convenient to implement the method for detecting AF and SA according to the disclosure at a resource-limited clinic or at home.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method of establishing a model for detecting atrial fibrillation (AF) and sleep apnea (SA), adapted to be implemented by a computer system that stores multiple training electrocardiograms, the method comprising steps of:
dividing each of the training electrocardiograms into multiple training segments, each of which contains a common length of time of recorded electrical activity of a heart;
for each of the training segments derived from each of the training electrocardiograms, labeling, based on a user input, the training segment with a symptom indicator that indicates whether the training segment is related to AF and whether the training segment is related to SA;
for each of the training segments derived from each of the training electrocardiograms, performing feature extraction on the training segment to obtain an entry of feature data; and
establishing the model for detecting AF and SA by using a machine learning algorithm based on the entries of feature data obtained respectively from the training segments that are derived from each of the training electrocardiograms and the symptom indicators labelled respectively on the training segments that are derived from each of the training electrocardiograms.

2. The method as claimed in claim 1, wherein for each of the training segments derived from each of the training electrocardiograms, the symptom indicator indicates one of a first condition that the training segment is related to both AF and SA, a second condition that the training segment is related to AF but not to SA, a third condition that the training segment is related to SA but not to AF, and a fourth condition that the training segment is related to neither AF nor SA.

3. The method as claimed in claim 1, further comprising a step of, prior to the step of dividing each of the training electrocardiograms into multiple training segments, performing zero-mean subtraction and normalization on the training electrocardiograms.

4. The method as claimed in claim 1, wherein for each of the training segments derived from each of the training electrocardiograms, the step of performing feature extraction on the training segment is to perform reconstruction independent component analysis (RICA) on the training segment.

5. The method as claimed in claim 1, wherein the model for detecting AF and SA has an input layer, sixteen convolution layers, fifteen batch normalization layers, fifteen leaky rectified linear unit (ReLU) activation layers, five max pooling layers, a global average pooling (GAP) layer, a softmax layer and an output layer.

6. A method for detecting atrial fibrillation (AF) and sleep apnea (SA), adapted to be implemented by a computer system that stores a model for detecting AF and SA, the method comprising steps of:
obtaining a target electrocardiogram that is related to a subject;
dividing the target electrocardiogram into multiple target segments, each of which contains a common length of time of recorded electrical activity of a heart of the subject;
for each of the target segments derived from the target electrocardiogram, performing feature extraction on the target segment to obtain an entry of feature data; and
for each of the target segments, obtaining a symptom prediction result by using the model for detecting AF and SA based on the entry of feature data obtained from the target segment, the symptom prediction result indicating whether the target segment is related to AF and indicating whether the target segment is related to SA.

7. The method as claimed in claim 6, wherein the step of performing feature extraction on the target segment is to perform reconstruction independent component analysis (RICA) on the target segment.

8. The method as claimed in claim 6, wherein the step of obtaining a symptom prediction result is to classify the target segment into one of a first medical condition which is related to both AF and SA, a second medical condition which is related to AF but not to SA, a third medical condition which is related to SA but not to AF, and a fourth medical condition which is related to neither AF nor SA, and to obtain the symptom prediction result indicating one of the first to fourth medical conditions into which the target segment is classified.

9. The method as claimed in claim 8, further comprising, subsequent to the step of determining whether the target segment is related to AF and determining whether the target segment is related to SA, a step of:
obtaining a disease risk assessment result for the subject based on the symptom prediction results respectively of the target segments, the disease risk assessment result indicating whether the subject is at risk of having AF and indicating whether the subject is at risk of having SA.

10. The method as claimed in claim 9, wherein the step of obtaining a disease risk assessment result includes sub-steps of:
for each of the first to fourth medical conditions, counting a number of the target segments that are classified into the medical condition so as to obtain four numbers respectively for the first to fourth medical conditions;
selecting one of the first to fourth medical conditions that corresponds to a greatest one of the four numbers; and
obtaining the disease risk assessment result to indicate said one of the first to fourth medical conditions.

11. The method as claimed in claim 6, wherein the model for detecting AF and SA has an input layer, sixteen convolution layers, fifteen batch normalization layers, fifteen leaky rectified linear unit (ReLU) activation layers, five max pooling layers, a global average pooling (GAP) layer, a softmax layer and an output layer.
